(19) United States
(12) Patent Application Publication
(10) Pub. No.: US 2019/0183459 A1
(43) Pub. Date: Jun. 20, 2019

(54) ULTRASONIC DIAGNOSTIC DEVICE

(71) Applicant: HITACHI, LTD., Tokyo (JP)

(72) Inventors: Wataru SAWADA, Tokyo (JP); Kazuya MOTOKI, Tokyo (JP); Toru WATANABE, Tokyo (JP)

(73) Assignee: HITACHI, LTD., Tokyo (JP)

(21) Appl. No.: 16/326,684

(22) PCT Filed: Mar. 22, 2017
(86) PCT No.: PCT/JP2017/011495

§ 371 Date: Feb. 19, 2019

(30) Foreign Application Priority Data
Aug. 24, 2016 (JP) 2016-163328

(51) Int. Cl. A61B 8/00; A61B 8/14

(52) U.S. Cl. CPC A61B 8/4444; A61B 8/56; A61B 8/461; A61B 8/14

(57) ABSTRACT

A probe 12 includes a laminated block 24 in which a protective layer 50, an acoustic matching layer 52, a vibrator array 20, a backing member 54, a relay board 56, and an IC 22 are laminated. A temperature sensor unit 26 is provided on a side surface of the laminated block 24. The temperature sensor unit 26 is provided with a metal film 60 provided in the vicinity of an edge on the front-side (the transmission/reception surface side) of the side surface of the laminated block 24, so as to extend along the front-side edge, and a thermistor 62 to detect a temperature of the metal film 60. The metal film 60 exerts a detection region expansion function of expanding a temperature detection region of the thermistor 62 in a direction where a temperature gradient occurs in the wave transmission/reception surface of the probe 12. Further, the metal film 60 also exerts a thermal diffusion function of diffusing heat of the wave transmission/reception surface of the probe 12.

### TECHNICAL FIELD

The present invention relates to an ultrasonic diagnostic device and an ultrasonic probe, and more particularly, to a technique for management of the surface temperature of an ultrasonic wave transmission/reception surface.

### BACKGROUND ART

An ultrasonic diagnostic device is a device to transmit/receive an ultrasonic wave with respect to a subject and form an ultrasonogram based on a reception signal obtained by the transmission/reception. The ultrasonic diagnostic device has an ultrasonic probe to transmit/receive an ultrasonic wave. The ultrasonic probe is provided with a vibrator array which is formed with plural vibration elements and which transmits/receives an ultrasonic wave, an electronic circuit to supply a transmission signal to the vibrator array or to process a reception signal from the vibrator array, and the like. The electronic circuit is provided for channel reduction processing to reduce the number of wirings included in a cable connecting the ultrasonic probe to the device main body, and the like. When the vibrator array and the electronic circuit operate, heat generation occurs in these parts.

The ultrasonic probe is used in a status where it is held to the subject. Accordingly, to prevent the subject from invasion with heat occurred from the ultrasonic probe, it is necessary to appropriately manage the temperature of a surface where the ultrasonic wave is transmitted/received and brought into contact with the subject (hereinbelow, described as “wave transmission/reception surface”). There are regulations regarding the temperature of the wave transmission/reception surface. For example, the temperature of the wave transmission/reception surface is specified in IEC (International Electrotechnical Commission) that it shall not exceed 43° C. in normal use status.

Conventionally, the temperature of the wave transmission/reception surface is managed by providing the ultrasonic probe with a temperature sensor.

For example, patent literature 1 discloses an ultrasonic diagnostic device having an ultrasonic prove including a vibrator array, an electronic circuit as a main heat source, and a relay board provided between the vibrator array and the electronic circuit. In the ultrasonic probe, a temperature sensor is provided on the relay board in the vicinity of the electronic circuit as the main heat source. The temperature of the wave transmission/reception surface is estimated based on a detection temperature from the temperature sensor and ultrasonic wave transmission/reception conditions. Further, patent literature 2 discloses an ultrasonic diagnostic device in which two temperature sensors are embedded in a backing member provided on the rear side (opposite side to the contact surface with respect to the subject) of the vibrator array. The temperature of the wave transmission/reception surface is calculated based on detection temperatures from the two temperature sensors.

### CITATION LIST

### Patent Literature

Patent Literature 1: Japanese Patent No. 5771294
Patent Literature 2: Japanese Patent No. 5099681

### SUMMARY OF INVENTION

### Technical Problem

In some cases, the temperatures in the respective positions in the wave transmission/reception surface are not constant, i.e., a temperature gradient occurs in the wave transmission/reception surface. For example, in some cases, the heat dissipation at the center of the wave transmission/reception surface is lower in comparison with its end. In such a case, the temperature at the center is higher than that at the end. Otherwise, in some cases, an ultrasonic wave is transmitted from a part of the vibration elements in the vibrator array provided in the ultrasonic probe as in the case of Doppler inspection. In such a case, in the wave transmission/reception surface, the temperature in a position in the vicinity of the vibration elements used for the transmission particularly rises. When a temperature gradient occurs in the wave transmission/reception surface, it is necessary to prevent the maximum temperature in the wave transmission/reception surface from exceeding the regulated temperature.

As in the conventional techniques, when the temperature sensor is provided in a position comparatively away from the wave transmission/reception surface and the temperature of the wave transmission/reception surface is estimated based on the detection temperature from the temperature sensor, upon occurrence of temperature gradient in the wave transmission/reception surface, it is difficult to estimate the maximum temperature in the wave transmission/reception surface.

Note that to detect the temperature of the wave transmission/reception surface, the wave transmission/reception surface may be provided with the temperature sensor. Especially when the wave transmission/reception surface is provided with plural temperature sensors, even though a temperature gradient occurs in the wave transmission/reception surface, it is possible to detect the maximum temperature in the wave transmission/reception surface. However, when the wave transmission/reception surface is provided with the temperature sensor(s), there is a fear of influence on ultrasonic wave transmission/reception performance. Accordingly, it is not appropriate to provide the wave transmission/reception surface with the temperature sensor(s).

The object of the present invention is to estimate the maximum temperature in the wave transmission/reception surface with high accuracy even when a temperature gradient occurs in the wave transmission/reception surface of an ultrasonic probe.

### Solution to Problem

An ultrasonic diagnostic device according to the present invention comprises: an ultrasonic probe having: a laminated body including a vibrator array to transmit/receive an ultrasonic wave, an acoustic matching layer provided between an ultrasonic wave transmission/reception surface and the vibrator array, and a backing layer; and a temperature detection unit provided on at least one side surface of the laminated body; and a temperature estimation unit that estimates a surface temperature of the wave transmission/reception surface based on a detection temperature detected with the temperature detection unit, wherein the temperature detection unit includes: a heat conduction member to receive heat from the laminated body, which is provided in the vicinity of an edge on the wave transmission/reception surface side of the side surface and which has a shape extending along the edge; and a temperature sensor to detect a temperature of the heat conduction member. Desirably, the heat conduction member exerts a detection region expansion function of expanding a temperature detection region of the temperature sensor, and a thermal diffusion function of diffusing heat of the wave transmission/reception surface.

According to the above configuration, the heat conduction member is provided in the vicinity of the edge of the side surface of the laminated body on the wave transmission/reception surface side. That is, it is provided in the vicinity of the wave transmission/reception surface. In this configuration, the heat of the wave transmission/reception surface is transmitted via the laminated body to the heat conduction member, and the transmitted heat is detected with the temperature sensor. As the heat conduction member extends along the edge of the side surface of the laminated body on the wave transmission/reception surface side, even though a temperature gradient occurs in the wave transmission/reception surface, the heat is transmitted from a maximum temperature position in the wave transmission/reception surface to the heat conduction member. The heat is detected with the temperature sensor. With this configuration, even when the maximum temperature position is changed in the wave transmission/reception surface, the temperature sensor appropriately detects the maximum temperature in the wave transmission/reception surface. In this manner, the heat conduction member has a detection region expansion function of expanding the temperature detection region of the temperature sensor. Further, the heat conduction member also exerts a thermal diffusion function of diffusing the heat of the wave transmission/reception surface. With the thermal diffusion function, it is possible to lower the temperature of the wave transmission/reception surface.

Desirably, the heat conduction member extends in a strip in a direction along the edge.

From the viewpoint of the thermal diffusion function, it is more advantageous that the area of the heat conduction member is widened. However, in such a case, there is a possibility that the heat conduction member transmits the heat from a heat source provided in a position away from the wave transmission/reception surface to the wave transmission/reception surface side. Accordingly, the heat conduction member has a belt shape along the edge of the side surface of the laminated body on the wave transmission/reception surface side. That is, by keeping a comparatively long distance as the distance from the heat source to the heat conduction member, it is possible to cause the heat conduction member to exert the detection region expansion function while suppressing transmission of the heat from the heat source to the wave transmission/reception surface side. Note that even though the heat conduction member has a belt shape, it also exerts the thermal diffusion function of diffusing the heat in the direction along the edge of the side surface of the laminated body on the wave transmission/reception surface side.

Desirably, the edge on the wave transmission/reception surface side of the side surface is curved, and an end at least on the wave transmission/reception surface side of the heat conduction member has a curved shape along the edge on the wave transmission/reception surface side of the side surface.

When the ultrasonic probe is a convex type probe, the edge of the side surface of the laminated body on the wave transmission/reception surface side is curved. In accordance with the curvature, the end of the heat conduction member on the wave transmission/reception surface side has a curved shape. Accordingly, it is possible to entirely further reduce the distance between the wave transmission/reception surface and the heat conduction member. With this configuration, the heat conduction member more appropriately receives the heat from the wave transmission/reception surface via the laminated body.

Desirably, the heat conduction member is formed with a metal film. Further, desirably, the temperature detection unit is a board connected to the heat conduction member, and further comprises a board including a thermal conduction path to conduct heat from the heat conduction member to the temperature sensor, and the temperature sensor is provided on the board.

Further, an ultrasonic probe according to the present invention comprises: a laminated body including a vibrator array to transmit/receive an ultrasonic wave, an acoustic matching layer provided between an ultrasonic wave transmission/reception surface and the vibrator array, and a backing layer; and a temperature detection unit provided at least on one side surface of the laminated body, wherein the temperature detection unit includes: a heat conduction member to receive heat from the laminated body, which is provided in the vicinity of an edge on the wave transmission/reception surface side of the side surface, and which has a shape extending along the edge; and a temperature sensor to detect a temperature of the heat conduction member.

### Advantageous Effects of Invention

According to the present invention, even when temperature gradient occurs in the wave transmission/reception surface of the ultrasonic probe, it is possible to estimate the maximum temperature in the wave transmission/reception surface with high accuracy.

### BRIEF DESCRIPTION OF DRAWINGS

FIG. 1 is a schematic configuration diagram of an ultrasonic diagnostic device according to a present embodiment;

FIG. 2 is a diagram showing a thermal network;

FIG. 3 is a perspective view of a laminated block and a temperature sensor unit in a first embodiment;

FIG. 4 is an exploded perspective view of the laminated block and the temperature sensor unit in the first embodiment;

FIG. 5 is a side view of the laminated block and the temperature sensor unit in the first embodiment;

FIG. 6 is a cross-sectional view of the laminated block and the temperature sensor unit in the first embodiment;

FIG. 7A is a side view of a laminated block 24 according to the present embodiment;

FIG. 7B is a diagram showing thermistor detection temperatures in respective maximum temperature positions in the first embodiment;

FIG. 8A is a side view of the laminated block 24 when a thermistor 62 is provided in the same position as that in the present embodiments but a metal film 60 is not provided;

FIG. 8B is a diagram showing the thermistor detection temperatures in the respective maximum temperature positions when the metal film is not used;

FIG. 9A is a side view of the laminated block 24 when the thermistor 62 is provided on a relay board 56 without providing the metal film 60;

FIG. 9B is a diagram showing the thermistor detection temperatures in the respective maximum temperature positions when the thermistor is provided on the relay board;

FIG. 10 is a perspective view of the laminated block and the temperature sensor unit in a second embodiment;

FIG. 11 is an exploded perspective view of the laminated block and the temperature sensor unit in the second embodiment;

FIG. 12 is a side view of the laminated block and the temperature sensor unit in the second embodiment; and

FIG. 13 is a cross-sectional view of the laminated block and the temperature sensor unit in the second embodiment.

### DESCRIPTION OF EMBODIMENTS

Hereinbelow, embodiments of an ultrasonic diagnostic device according to the present invention will be described.

### First Embodiment

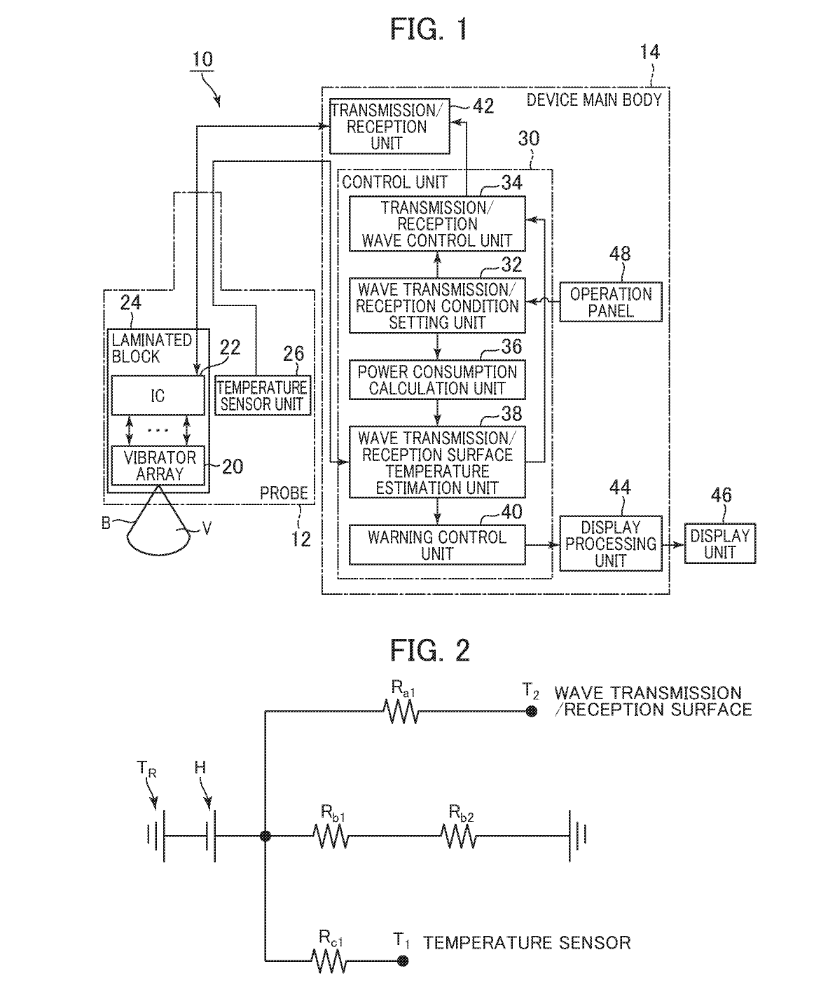

FIG. 1 is a schematic configuration diagram of an ultrasonic diagnostic device 10 according to the present embodiment. The ultrasonic diagnostic device 10 is a medical device which is generally installed in a medical institution such as a hospital and which performs ultrasonic wave diagnosis with respect to a living body. The ultrasonic diagnostic device 10 includes a probe 12 as an ultrasonic probe to transmit/receive an ultrasonic wave and a device main body 14.

The probe 12 is an ultrasonic probe which is held to a subject's surface to perform ultrasonic wave transmission/reception. The probe 12 is communicably connected to the device main body 14 with a cable or via radio communication. In the present embodiment, the probe 12 is a convex type probe. However, the probe 12 may be any other type of probe.

A vibrator array 20 is formed with one-dimensionally or two-dimensionally arrayed plural vibration elements. The respective vibration elements are formed of monocrystal, e.g. ceramics such as PZT (lead zirconate titanate) or PMT-PT (lead magnesium niobate-lead titanate solid solution). When a driving signal is supplied from the device main body 14 via an IC 22 to the respective vibration elements, the respective vibration elements vibrate and an ultrasonic beam is transmitted. Further, the respective vibration elements receive a reflection echo reflected from the subject. The vibrator array 20 outputs a reception signal via the IC 22 to the device main body 14 based on the received reflection echo. The vibrator array 20 operates and generates heat when supplied with electric power. The power consumption and heat generation amount in the vibrator array 20 vary in correspondence with ultrasonic wave transmission/reception condition, e.g. diagnostic mode, transmission voltage, wavenumber, pulse interval time (PRT), and frequency.

The IC 22 is an electronic circuit, e.g., an ASIC (Application Specific Integrated Circuit) where plural function circuits are united as one. The IC 22 in the present embodiment functions as a transmission sub-beam former and a reception sub-beam former. The vibration elements forming the vibrator array 20 are divided in plural groups. The transmission sub-beam former generates plural transmission signals having delay relationship by group based on a group transmission signal. The reception sub-beam former performs phasing addition processing with respect to the plural reception signals by group and generates a group reception signal by group. The plural group reception signals are processed with a transmission/reception unit 42 (described later) in the device main body 14, and become one beam data. With the above-described processing, the number of signal lines between the probe 12 and the device main body 14 is reduced. The IC 22 operates and generates heat when supplied with electric power. As in the case of the vibrator array 20, the power consumption and the heat generation amount in the IC 22 vary in correspondence with ultrasonic wave transmission/reception conditions. Note that the heat generation amount in the IC 22 is about 10 times of the heat generation amount in the vibrator array 20. The main heat generation source in the probe 12 is the IC 22.

In the probe 12, the vibrator array 20, the IC 22, and other members to be described later are laminated, and they form a laminated block 24 as a laminated body. The details of the laminated block 24 will be described later.

The probe 12 is provided with a temperature sensor unit 26 as a temperature detection unit. The temperature sensor unit 26 is provided with a temperature sensor for calculation of the temperature of the wave transmission/reception surface of the probe 12. As described above, since it is not possible to provide the wave transmission/reception surface of the probe 12 with the temperature sensor, the temperature sensor unit 26 is installed on a side surface of the laminated block 24. Temperature information indicating a detection temperature from the temperature sensor of the temperature sensor unit 26 is transmitted to the device main body 14. In the device main body 14, calculation to estimate the temperature of the wave transmission/reception surface of the probe 12 is performed based on the temperature information. The details of the temperature sensor unit 26 will be described later.

Next, the respective parts of the device main body 14 will be described.

A control unit 30 includes e.g. a microcontroller or a CPU (Central Processing Unit). The control unit 30 controls the respective parts of the ultrasonic diagnostic device 10 in accordance with a program stored in a memory (not shown) of the device main body 14. With the program, the control unit 30 also functions as a wave transmission/reception condition setting unit 32, a transmission/reception wave control unit 34, a power consumption calculation unit 36, a wave transmission/reception surface temperature estimation unit 38 as a temperature estimation unit, and a warning control unit 40.

The wave transmission/reception condition setting unit 32 sets ultrasonic wave transmission/reception conditions in the vibrator array 20. The wave transmission/reception conditions are set based on an operator's instruction. For example, the operator uses an operation panel 48 to be described later to select a desired heat generation mode from prepared plural heat generation modes. In the present embodiment, three modes, “Low”, “Mid”, and “High” are prepared as the heat generation modes. The heat generation mode “High” is a mode to make the best of the performance of the ultrasonic diagnostic device. It is possible to obtain an ultrasonogram with high resolution and high response. On the other hand, the heat generation amount in the vibrator array 20 and the IC 22 is the maximum among the three modes. The heat generation mode “Low” is a mode to reduce e.g. the transmission voltage or wavenumber of the ultrasonic wave, or to increase PRT. The image quality of the ultrasonogram is degraded, on the other hand, the heat generation amount in the vibrator array 20 and the IC 22 is the minimum in the three modes. The heat generation mode “Mid” is an intermediate mode between these modes. The wave transmission/reception condition setting unit 32 sets wave transmission/reception conditions corresponding to an input heat generation mode. The wave transmission/reception conditions include vibration elements driving voltage, frequency, wavenumber, pulse interval time (PRT), diagnostic mode and the like.

The transmission/reception wave control unit 34 controls the transmission/reception unit 42 based on the wave transmission/reception conditions set with the wave transmission/reception condition setting unit 32, to operate the vibrator array 20 and the IC 22 under the wave transmission/reception conditions. Further, the transmission/reception wave control unit 34 controls the transmission/reception unit 42 based on a signal from the wave transmission/reception surface temperature estimation unit 38 to be described later, to stop ultrasonic wave transmission/reception in the probe 12. For example, when information indicating that the temperature of the wave transmission/reception surface of the probe 12 is a predetermined temperature is received from the wave transmission/reception surface temperature estimation unit 38, the transmission/reception wave control unit 34 controls the transmission/reception unit 42, to immediately stop the ultrasonic wave transmission/reception.

The power consumption calculation unit 36 calculates power consumption in the vibrator array 20 and the IC 22 based on the wave transmission/reception conditions set with the wave transmission/reception condition setting unit 32. The calculation of power consumption may be performed by using a function indicating the relationship between the wave transmission/reception condition and power consumption. Otherwise, it may be configured such that the correspondence between wave transmission/reception conditions and power consumption is previously stored in the form of table, and the power consumption is specified based on the wave transmission/reception conditions and the table.

The wave transmission/reception surface temperature estimation unit 38 estimates the temperature of the wave transmission/reception surface of the probe 12 based on the power consumption calculated with the power consumption calculation unit 36 and the temperature information sent from the probe 12. Hereinbelow, the details of the processing with the wave transmission/reception surface temperature estimation unit 38 will be described with reference to FIG. 2.

FIG. 2 is a diagram showing a thermal network. The relationship among temperature difference, heat flow, and thermal resistance among objects is similar to the relationship among voltage, current, and resistance in an electric circuit. Accordingly, it is possible to represent temperature difference, heat flow, and thermal resistance among objects as a thermal network similar to an electric circuit. The thermal network shown in FIG. 2 includes a reference temperature TR which is the same symbol as that of GND in an electric circuit, a heat source H as the same symbol of a direct-current power source in the electric circuit, and a thermal resistance R as the same symbol of the resistance in the electric circuit. For the sake of simplification, the thermal network in FIG. 2 has one heat source.

In the thermal network in FIG. 2, the reference temperature TR is an ambient temperature. A heat source H is e.g. the vibrator array 20 or the IC 22. The thermal resistance R is a value representing the degree of difficulty in temperature transmission in the respective objects, and the unit is (° C./W). For example, when the heat source H is the IC 22, a thermal resistance Ra1 provided between the heat source H and a wave transmission/reception surface node indicates a synthesized resistance of the thermal resistances in the plural objects between the IC 22 and the wave transmission/reception surface. The thermal resistances in the respective objects are previously obtained by experiment or the like.

It is possible to estimate a temperature T2 of the wave transmission/reception surface node based on the following expression.

T2=T1+Tdif+D (Expression 1)

In the expression 1, T1 indicates a detection temperature from the temperature sensor (temperature sensor node temperature); Tdif, the temperature difference between the temperature of the temperature sensor node and the wave transmission/reception surface node; and D, a first order lag element indicating time difference until the heat from the heat source H arrives at the wave transmission/reception surface node.

Tdif is calculated with the following expression 2.

Tdif=α×W (Expression 2)

In the expression 2, α is a value obtained by subtracting a thermal resistance value from the heat source H to the wave transmission/reception surface node, from a thermal resistance value from the heat source H to the temperature sensor node. For example, in the thermal network shown in FIG. 2, when a thermal resistance Rc1 from the heat source H to the temperature sensor node is 2(° C./W), and a thermal resistance Ra1 from the heat source H to the wave transmission/reception surface node is 6(° C./W), α is 2−6=−4. Further, in the expression 2, a power consumption W is the power consumption (W) of the heat source H. Accordingly, e.g., when the power consumption of the IC 22 as the heat source H is 1 W, Tdif is calculated as −4×1=−4(° C.).

The first order lag element D is calculated with the following expression 3.

D=β×exp(−Δt/K) (Expression 3)

In the expression 3, β is a variable which varies in correspondence with power consumption of the heat source H. Further, Δt is elapsed time (sec) from the start of ultrasonic wave transmission/reception; and K, a time constant.

When plural heat sources H exist in the thermal network, the temperature difference between the temperature of the temperature sensor node and the temperature of the wave transmission/reception surface node is calculated by heat source H, and as Tdif in the expression 1, a value obtained by summing up the values regarding the respective heat sources is used. For example, a thermal resistance value from the first heat source to the wave transmission/reception surface node is subtracted from a thermal resistance value from the first heat source to the temperature sensor node, as α1. Then a value α1W1 is obtained by multiplying the value a1 with the first heat source power consumption W1. Next, a thermal resistance value from the second heat source to the wave transmission/reception surface node is subtracted from a thermal resistance value from the second heat source to the temperature sensor node, as α2. Then a value α2W2 is calculated by multiplying a2 with the second heat source power consumption W2. Then, the value obtained by a1W1+a2W2 is used as Tdif in the expression 1.

Information corresponding to the thermal network indicating heat transmission in the probe 12 is stored in the memory of the device main body 14. The wave transmission/reception surface temperature estimation unit estimates the temperature of the wave transmission/reception surface of the probe 12 based on the information corresponding to the thermal network, the temperature information sent from the probe 12 (detection temperature from the temperature sensor), and the power consumption of the heat source (the vibrator array 20 and the IC 22) calculated with the power consumption calculation unit 36.

The wave transmission/reception surface node in the thermal network indicates a predetermined position of the wave transmission/reception surface of the probe 12. For example, when the wave transmission/reception surface node indicates a central position of the wave transmission/reception surface of the probe 12, the thermal resistance Ra1 indicates the thermal resistance from the heat source H to the central position of the wave transmission/reception surface of the probe 12. This positional relationship is the same regarding the heat source H. The heat source H in the thermal network indicates a predetermined position of the heat source. Accordingly, e.g., assuming that the heat source H is the vibrator array 20, even when a part of the vibration elements of the vibrator array 20 are substantially the heat source H, as in the case of Doppler inspection, as the heat source H in the thermal network, a predetermined position (e.g., the central position) of the vibrator array 20 is indicated. It may be configured such that the thermal network as shown in FIG. 2 is constructed by each position of the wave transmission/reception surface of the probe 12 or by each position of the heat source. In this case, it is considered that even when a temperature gradient occurs in the wave transmission/reception surface of the probe 12, it is possible to appropriately estimate temperatures in the respective positions of the wave transmission/reception surface of the probe 12. However, to realize such estimation, huge information amount for storage in the device main body 14 or huge operation amount in the wave transmission/reception surface temperature estimation unit 38 are required, thus it is substantially impossible to realize it. According to the present embodiment, by providing the temperature sensor unit 26 (the details will be described later), without huge information amount or huge operation amount, even when a temperature gradient occurs in the wave transmission/reception surface of the probe 12, it is possible to appropriately detect a maximum temperature in the wave transmission/reception surface of the probe 12.

The warning control unit 40 performs control to output warning to promote the operator (user) of the ultrasonic diagnostic device 10 to lower the temperature of the wave transmission/reception surface based on the temperature of the wave transmission/reception surface estimated with the wave transmission/reception surface temperature estimation unit 38. When the estimated temperature of the wave transmission/reception surface is equal to or higher than a previously determined first threshold value and lower than a previously determined second threshold value, the warning control unit 40 outputs warning. In the present embodiment, 41° C. is determined as the first threshold value, and 43° C. is determined as the second threshold value. That is, in the present embodiment, when the estimated temperature of the wave transmission/reception surface is equal to or higher than 41° C. and lower than 43° C., the warning is outputted. The warning may be displayed as a warning message on a display unit 46, or emitted as sound, light or the like. It goes without saying that the warning is made by a combination of these means.

Note that when the estimated temperature of the wave transmission/reception surface is equal to or higher than the second threshold value, the wave transmission/reception surface temperature estimation unit 38 immediately performs control to stop the ultrasonic wave transmission/reception. When the estimated temperature of the wave transmission/reception surface is lower than the first threshold value, the wave transmission/reception surface temperature estimation unit 38 and the warning control unit 40 do not perform the ultrasonic wave transmission/reception stop processing or warning processing.

The transmission/reception unit 42 functions as a main transmission/reception beam former. The transmission/reception unit 42 receives a signal from the transmission/reception wave control unit 34, and supplies plural signals to drive the plural vibration elements of the vibrator array 20 to the IC 22. Further, the transmission/reception unit 42 receives plural reception signals via the IC 22 from the vibrator array 20. The plural reception signals are sent to an ultrasonogram forming unit (not shown) provided in the device main body 14. In the ultrasonogram forming unit, an ultrasonogram based on the reception signals is formed.

A display processing unit 44 performs control to display the temperature of the wave transmission/reception surface of the probe 12 estimated with the wave transmission/reception surface temperature estimation unit 38 on the display unit 46. It is preferable that the surface temperature is displayed in a realtime manner. Further, when an instruction is received from the warning control unit 40, control is performed to display a warning message on the display unit 46. Note that the display processing unit 44 also performs control to display the ultrasonogram formed with the ultrasonogram forming unit on the display unit 46.

The schematic configuration of the ultrasonic diagnostic device 10 is as described above. Hereinbelow, the details of the laminated block 24 and the temperature sensor unit 26 included in the probe 12 will be described.

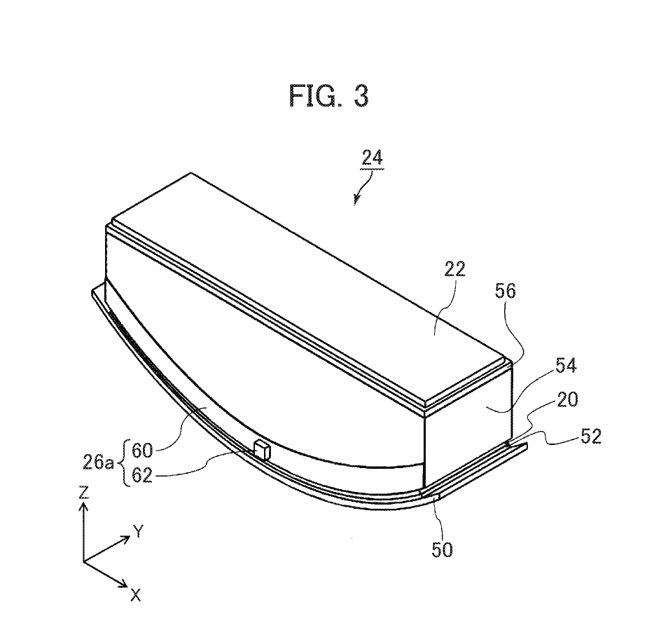

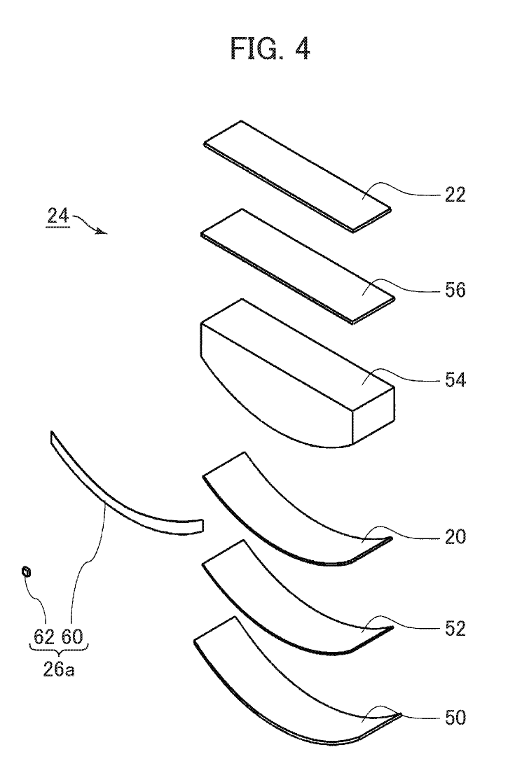

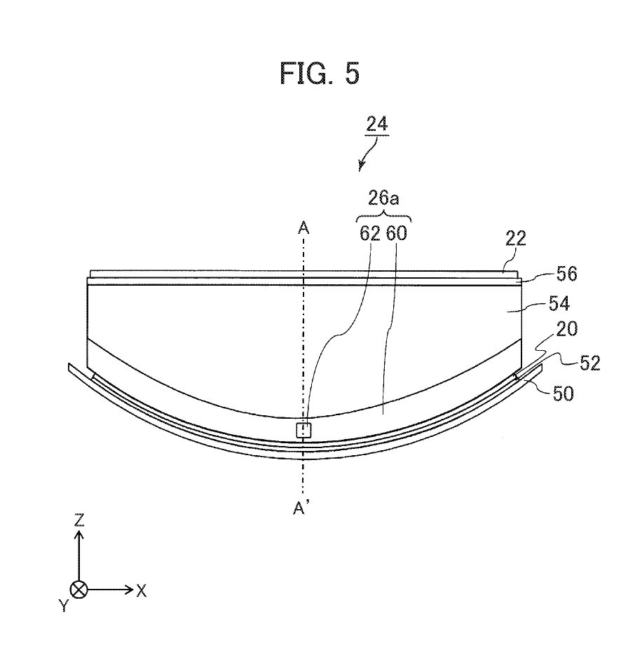

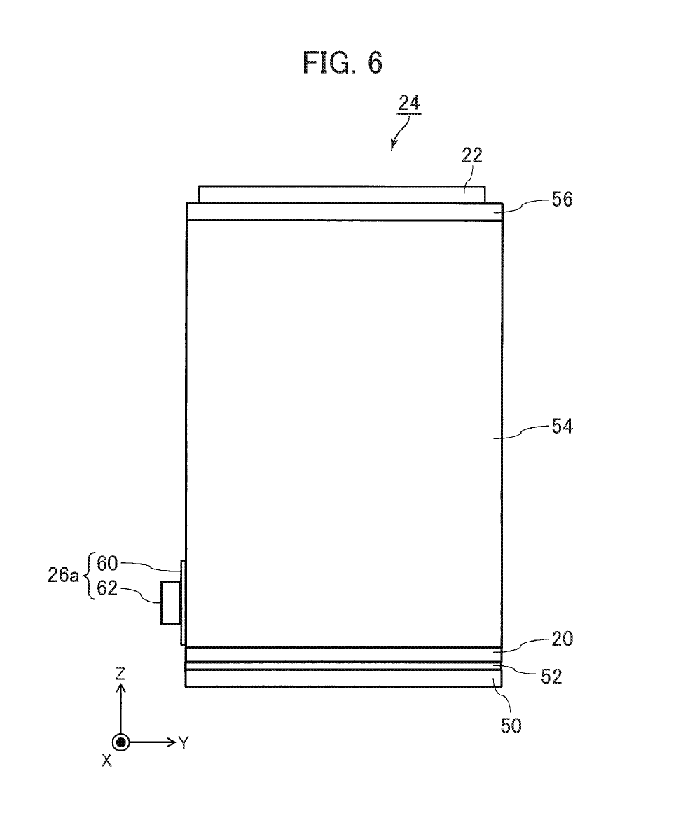

FIG. 3 shows a perspective view of the laminated block 24 and a temperature sensor unit 26a in the first embodiment. FIG. 4 shows an exploded perspective view of the laminated block 24 and the temperature sensor unit 26a. FIG. 5 shows a side view of the laminated block 24 and the temperature sensor unit 26a. FIG. 6 shows an A-A′ cross-sectional view in FIG. 5. As shown in FIG. 3 to FIG. 6, a lamination direction of the laminated block 24 is a z-axis, a direction orthogonal to the z-axis and in a longitudinal direction of the laminated block 24, an x-axis, and a lateral direction, a y-axis. Further, in the following description, the z-axis negative direction side is described as “front side”, while the z-axis positive direction side, “rear side”. Note that the front side is the wave transmission/reception surface side.

As shown in FIG. 3 to FIG. 6, in a plan view, the laminated block 24 is substantially rectangular parallelepiped with the x-axis direction in the longitudinal direction and the y-axis direction in the lateral direction. Further, as shown in FIG. 5, as the probe 12 is a convex type probe, in a side view, the front end of the laminated block 24 is curved.

The laminated block 24 is formed by laminating, from the front side, a protective layer 50, an acoustic matching layer 52, the vibrator array 20, a backing member 54 forming a backing layer, the relay board 56, and the IC 22. Note that in the present embodiment, the laminated block 24 includes the above-described respective layers. As a laminated body, it is formed with at least the acoustic matching layer 52, the vibrator array 20, and the backing member 54.

The protective layer 50 protects the layers from the acoustic matching layer 52. The protective layer 50 is formed of e.g. silicone rubber. As the probe 12 according to the present embodiment is a convex type probe, the vibrator array 20 has a curved shape (semi-cylindrical shape). In accordance with the shape, the protective layer 50 is also curved. The front-side surface of the protective layer 50 (lower surface in the example in FIG. 3 and FIG. 4) is the surface brought into contact with the subject, i.e., the wave transmission/reception surface.

The acoustic matching layer 52 is provided between the protective layer 50 and the vibrator array 20, for suppressing ultrasonic wave reflection by acoustic impedance matching between the vibrator array 20 and the subject. The acoustic matching layer 52 is formed of e.g. resin, carbon, or carbon. The acoustic matching layer 52 may be formed with one or more layers.

The backing member 54 suppresses unnecessary vibration of the vibrator array 20. The backing member 54 is formed of material with high acoustic impedance such as resin. Further, the backing member 54 according to the present embodiment has respective vibration elements included in the vibrator array 20 and plural conduction wires (leads) for electrical connection to the IC 22. The plural leads are provided along the z-axis. The front-side ends of the plural leads and the rear-side surfaces of the respective vibration elements are brought into contact, thus electrical conduction between the respective vibration elements and the plural leads is established. Note that as described above, the vibrator array 20 has a curved shape, accordingly, the front-side surface of the backing member 54 also has a semi-cylindrical curved shape.

The relay board 56 is a board to relay electrical connection between the vibrator array 20 and the IC 22. Plural pads are provided on the front-side surface of the relay board 56. The plural pads and the rear-side ends of the plural leads of the backing member 54 are brought into contact, thus electrical conduction is established between them. The IC 22 is mounted on the rear-side surface of the relay board 56. With this configuration, electrical conduction is established between the IC 22 and the respective vibration elements via the relay board 56 and the plural leads of the backing member 54. Further, although not shown in FIG. 3 to FIG. 6, the relay board 56 is connected to an FPC (Flexible Printed Circuits) for electrical connection between the IC 22 and the device main body 14.

The temperature sensor unit 26a is provided at least one side surface of the above-described laminated block 24. In the present embodiment, the temperature sensor unit 26a is provided on two side surfaces (long side surfaces) extending in the longitudinal direction of the laminated block 24. Since the configurations of the two temperature sensor units 26a provided on the both long side surfaces are the same, one temperature sensor unit 26a will be described here. The temperature sensor unit 26a includes a metal film 60 as a heat conduction member and a thermistor 62 as a temperature sensor.

The metal film 60 is a thin film or plate-shaped member formed of metal. The metal film 60 receives the heat of the wave transmission/reception surface of the probe 12 via the laminated block 24, and conducts the heat to the thermistor 62. It is preferable that the metal film 60 is formed of a material with high thermal conductivity. It is ideal that the temperature is uniform in the respective positions in the metal film 60. At least the metal film 60 is formed of a material with higher thermal conductivity than that of the respective members forming the laminated block 24. In the present embodiment, the metal film 60 is formed of gold.

The metal film 60 is provided in a position close to the wave transmission/reception surface as much as possible, on the side surface of the laminated block 24. That is, it is provided in the vicinity of the front-side (on the wave transmission/reception surface side) edge of the side surface of the laminated block 24. Further, the metal film 60 is provided so as to extend along the front-side edge of the side surface of the laminated block 24. As described above, the front-side edge of the side surface of the laminated block 24 has a curved shape. Accordingly, in accordance with the shape, at least the front-side end of the metal film 60 has a curved shape.

In the present embodiment, as shown in FIG. 3, FIG. 5 and FIG. 6, the metal film 60 is provided along the front-side edge of the backing member 54. It goes without saying that as long as the metal film 60 is appropriately provided in the probe 12, the metal film 60 may be provided on the side of the vibrator array 20, the acoustic matching layer 52, or the protective layer 50. Further, as shown in FIG. 3 and FIG. 5, the metal film 60 extends from one end of the backing member 54 in the longitudinal direction, along the front-side edge of the long side surface, to the other end in the longitudinal direction. Note that in the present embodiment, the metal film 60 is provided only on the long side surface of the backing member 54. However, the metal film 60 may extend to a short side surface of the backing member 54. Further, the metal film 60 may have a shape surrounding the side surface of the laminated block 24.

The resistance value of the thermistor 62 varies in correspondence with temperature change. Accordingly, the resistance value of the thermistor 62 indicates a temperature in the vicinity of the installation position of the thermistor 62. The thermistor 62 is attached to the metal film 60 to detect the temperature of the metal film 60. In the present embodiment, the thermistor 62 is provided at the center in the x-axis direction (longitudinal direction) on the metal film 60. Note that the position where the thermistor 62 is provided is not limited to that position. As long as the thermal conductivity of the metal film 60 is high and the thermal uniformity on the metal film 60 is substantially secured, the thermistor 62 may be provided in any position on the metal film 60. The metal film 60 may be at the ground potential, and one-side terminal of the thermistor 62 may be electrically connected to the metal film 60. The other-side terminal of the thermistor 62 is electrically connected via a wire material, the relay board 56, the FPC or the like, to the device main body 14. With this configuration, the resistance value of the thermistor 62 is detected with the device main body 14 (wave transmission/reception surface temperature estimation unit 38).

In the present embodiment, only one thermistor 62 is provided, however, in preparation for occurrence of temperature gradient in the metal film 60, plural thermistors 62 may be provided. The plural thermistors 62 are provided in plural positions of the metal film 60, and with this configuration, the temperatures in the plural positions of the metal film 60 are detected. As described later, when the maximum temperature position varies in the wave transmission/reception surface of the probe 12, to always appropriately estimate the maximum temperature in the wave transmission/reception surface, it is necessary that the difference between the maximum temperature and the detection temperature from the thermistor 62 is always constant. When plural thermistors 62 are provided, a representative detection temperature value of the plural detection temperatures from the plural thermistors 62, to obtain a constant difference with respect to the maximum temperature in the wave transmission/reception surface is used. As such a representative detection temperature value, the highest detection temperature among the plural detection temperatures, the lowest temperature, or a mean value of the plural detection temperatures or the like may be used. Further, when plural thermistors 62 are provided, by comparing the detection temperatures from the respective thermistors 62, it is possible to detect failure of the thermistor 62.

With the temperature sensor unit 26a, even when a temperature gradient occurs in the wave transmission/reception surface of the probe 12, the wave transmission/reception surface temperature estimation unit 38 appropriately estimates the maximum temperature in the wave transmission/reception surface of the probe 12. The mechanism is as follows. Note that generally, a temperature gradient in the wave transmission/reception surface occurs in the x-axis direction (longitudinal direction), but does not occur in the y-axis direction (lateral direction).

First, with the metal film 60 provided along the front-side edge of the long side surface of the backing member 54, i.e. along a direction in which a temperature gradient may occur in the wave transmission/reception surface, a temperature detection region of the thermistor 62 is expanded in a direction in which a temperature gradient occurs. For example, the case where the thermistor 62 is provided at the center in the x-axis direction on the metal film 60 as in the case of the present embodiment will be considered. When a part of the vibration elements of the vibrator array 20 is driven (hereinbelow, the region of the driven vibration elements will be described as “transmission opening”) as in the case of Doppler inspection, the position indicating the maximum temperature in the wave transmission/reception surface (hereinbelow described as “maximum temperature position”) is the position in the vicinity of the transmission opening. Accordingly, when the transmission opening is shifted to the x-axis direction, the maximum temperature position is also shifted to the x-axis direction. In such a case, the heat in the maximum temperature position shifted to the x-axis direction is conducted through the metal film 60 to the vicinity of the thermistor 62. That is, the thermistor 62, even provided at the center in the x-axis direction, detects the temperature in the maximum temperature position shifted to the x-axis direction. In this manner, the metal film 60 exerts the detection region expansion function of expanding the temperature detection region of the thermistor 62.

Along with the function, the metal film 60 receives the heat of the wave transmission/reception surface via the laminated block 24, and diffuses the received heat in the expanding direction of the metal film 60. With this configuration, the heat of the wave transmission/reception surface is lowered, or the temperature gradient in the wave transmission/reception surface is mitigated. That is, the metal film 60 also exerts the thermal diffusion function of diffusing the heat of the wave transmission/reception surface.

From the viewpoint of the thermal diffusion function, widening the area of the metal film 60 as much as possible is considered. That is, providing the metal film 60 so as to cover the entire side surface of the laminated block 24 is considered. However, as described above, as the main heat source in the probe 12 is the IC 22, when the metal film 60 is expanded to the vicinity of the IC 22, the metal film 60 is susceptible to the heat from the IC 22. There is a fear that the heat from the IC 22 is easily transmitted to the front side (the wave transmission/reception surface side). Accordingly, in the present embodiment, the metal film 60 has a shape to extend in a strip in a direction along the front-side edge of the backing member 54. With this configuration, the distance between the metal film 60 and the IC 22 is comparatively long, and the heat conduction from the IC 22 to the metal film 60 is suppressed.

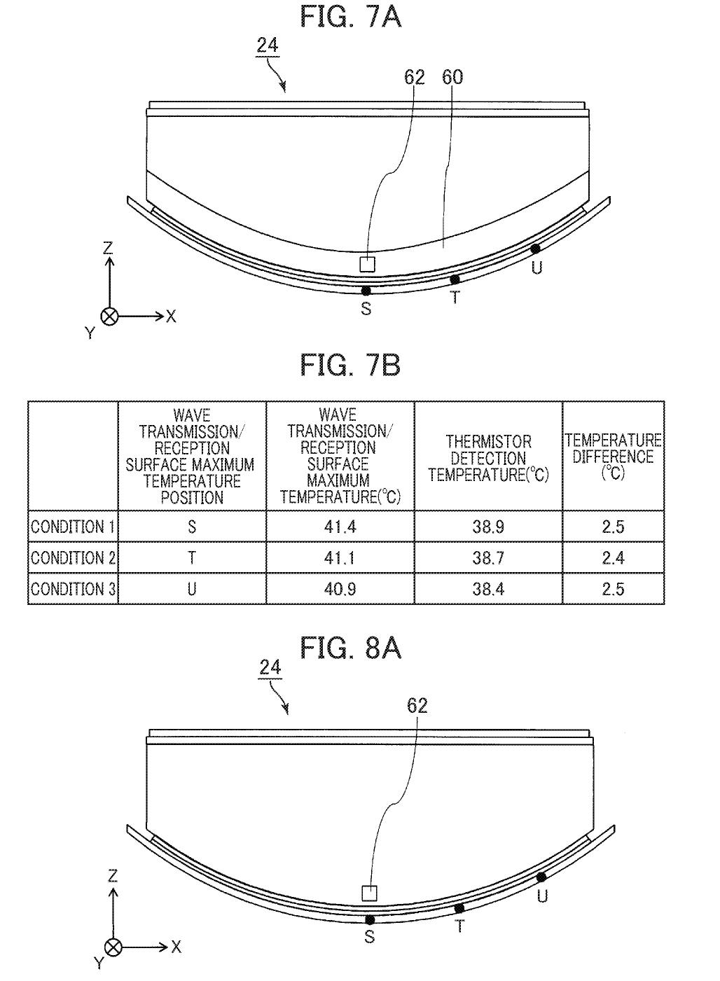

FIG. 7A and FIG. 7B show the detection temperatures from the thermistor 62 when the maximum temperature position in the wave transmission/reception surface is changed in the present embodiment.

FIG. 7A is a side view of the laminated block 24 according to the present embodiment. Here the maximum temperature position in the wave transmission/reception surface is changed by changing the position of the transmission opening. More particularly, the maximum temperature position in the wave transmission/reception surface when the central position of the transmission opening is the center in the x-axis direction (condition 1) is indicated with S. As heat generation occurs in the vibration elements included in the transmission opening, in the wave transmission/reception surface, a position corresponding to the central position of the transmission opening (the closest position) is the maximum temperature position S. Similarly, the maximum temperature position in the wave transmission/reception surface when the transmission opening is shifted to the x-axis direction by 10 mm (condition 2) is indicated with T. The maximum temperature position in the wave transmission/reception surface when the transmission opening is shifted to the x-axis direction by 20 mm (condition 3) is indicated with U. Note that in the above conditions 1 to 3, the position of the transmission opening differs, but the other ultrasonic wave transmission/reception conditions are the same.

FIG. 7B shows the maximum temperatures in the wave transmission/reception surface, the detection temperatures from the thermistor 62, and the temperature differences between the maximum temperatures in the wave transmission/reception surface and the detection temperatures from the thermistor 62 in the respective conditions 1 to 3. First, in the condition 1, i.e. when the maximum temperature position is S, the maximum temperature in the wave transmission/reception surface is 41.4° C., the detection temperature from the thermistor 62 is 38.9° C., and the temperature difference between them is 2.5° C. Further, in the condition 2, i.e. when the maximum temperature position is T, the maximum temperature in the wave transmission/reception surface is 41.1° C., the detection temperature from the thermistor 62 is 38.7° C., and the temperature difference between them is 2.4° C. In the condition 2, the distance between the maximum temperature position and the thermistor 62 is longer in comparison with the condition 1. As the heat from the maximum temperature position T is conducted through the metal film 60 to the vicinity of the thermistor 62, the temperature differences between the maximum temperatures in the wave transmission/reception surface and the detection temperatures from the thermistor are equivalent in the condition 1 and the condition 2. Further, in the condition 3, i.e. when the maximum temperature position is U, the maximum temperature in the wave transmission/reception surface is 40.9° C., the detection temperature from the thermistor 62 is 38.4° C., and the temperature difference between them is 2.5° C. Also in the condition 3, as the heat from the maximum temperature position U is conducted through the metal film 60 to the vicinity of the thermistor 62. Accordingly, when the condition 1 and the condition 3 are compared with each other, the temperature differences between the maximum temperatures in the wave transmission/reception surface and the detection temperatures from the thermistor are equivalent.

As described above, according to the present embodiment, even when any position in the wave transmission/reception surface of the probe 12 becomes the maximum temperature position, the temperature difference between the maximum temperature in the wave transmission/reception surface and the thermistor 62 is almost constant. With this configuration, as long as 2.5° C. is calculated as Tdif with the above-described expression 2, even when whatever position in the wave transmission/reception surface becomes the maximum temperature position, the maximum temperature in the wave transmission/reception surface is calculated with the above-described expression 1.

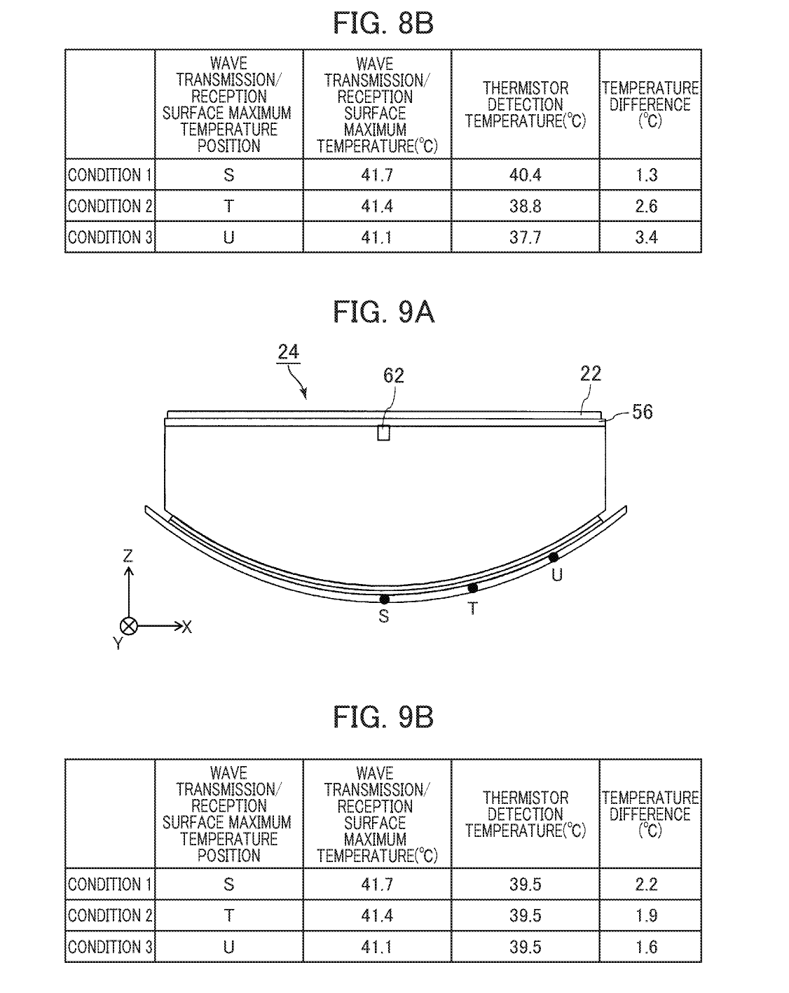

FIG. 8A and FIG. 8B show the detection temperatures from the thermistor 62 when the thermistor 62 is provided in the same position as that in the present embodiment but the metal film 60 is not provided, and the maximum temperature position in the wave transmission/reception surface is changed. It is possible to better grasp the effect of the metal film 60 by comparing FIG. 7A and FIG. 7B with FIG. 8A and FIG. 8B.

In the example in FIG. 8A and FIG. 8B, in the condition 1, i.e. when the maximum temperature position is S, the maximum temperature in the wave transmission/reception surface is 41.7° C., the detection temperature from the thermistor 62 is 40.4° C., and the temperature difference between them is 1.3° C. Further, in the condition 2, i.e. when the maximum temperature position is T, the maximum temperature in the wave transmission/reception surface is 41.4° C., the detection temperature from the thermistor 62 is 38.8° C., and the temperature difference between them is 2.6° C. In this manner, in the condition 1 and the condition 2, although the difference between the maximum temperatures in the wave transmission/reception surface is 0.3° C., the difference between the detection temperatures from the thermistor 62 is 1.6° C. The difference between the temperature differences is equal to or higher than 1° C. In the case of the condition 1, as the maximum temperature position S and the thermistor 62 are comparatively close to each other, the thermistor 62 detects a temperature close to the maximum temperature. In the case of the condition 2, as the distance between the maximum temperature position T and the thermistor 62 is longer in comparison with the condition 1, the thermistor 62 does not appropriately detect the temperature in the maximum temperature position T. In the condition 3, the maximum temperature in the wave transmission/reception surface and the detection temperature in the thermistor are further higher. In such a case, when Tdif is to be calculated with the above-described expression 2, it is necessary to adjust a such that Tdif becomes a greater value (e.g., 3.4° C. in the condition 3, or higher value) in consideration of safety. As a result, even when the transmission opening is at the center in the x-axis direction, the situation that the ultrasonic wave transmission/reception is stopped even though the temperature in the maximum temperature position S is about 39° C., is predicted. That is, it is not possible to sufficiently exert the performance of the ultrasonic diagnostic device.

Further, when FIG. 7A and FIG. 7B are compared with FIG. 8A and FIG. 8B, the maximum temperatures in the wave transmission/reception surface in the respective conditions are lower in FIG. 7A and FIG. 7B, i.e. when the metal film 60 is provided. This indicates that the temperature of the wave transmission/reception surface is lowered with the thermal diffusion function of the metal film 60.

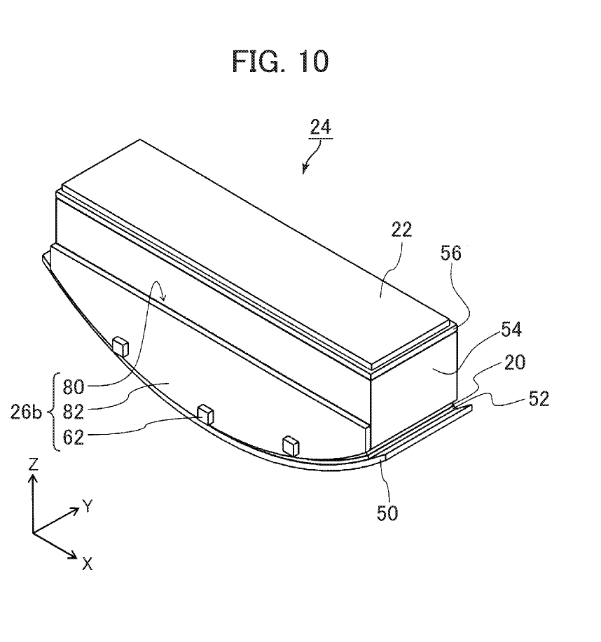

FIG. 9A and FIG. 9B show the detection temperatures from the thermistor 62 when the metal film 60 is not provided but the thermistor 62 is provided on the relay board 56. As described above, the main heat source in the probe 12 is the IC 22. Accordingly, when the thermistor 62 is provided on the relay board 56 in the vicinity of the IC 22, in the detection temperature from the thermistor 62, the heat from the IC 22 is dominant. The change of the maximum temperature position in the wave transmission/reception surface is not captured at all. Even in this case, as in the case of the example in FIG. 8A and FIG. 8B, when Tdif is to be calculated with the above-described expression 2, it is necessary to adjust a such that Tdif becomes higher value in consideration of safety. It is not possible to sufficiently exert the performance of the ultrasonic diagnostic device.

### Second Embodiment

Hereinbelow, a second embodiment will be described. In the second embodiment, only the configuration of the temperature sensor unit 26 is different in comparison with the above-described first embodiment. Accordingly, the explanations of the same parts as those in the first embodiment will be omitted.

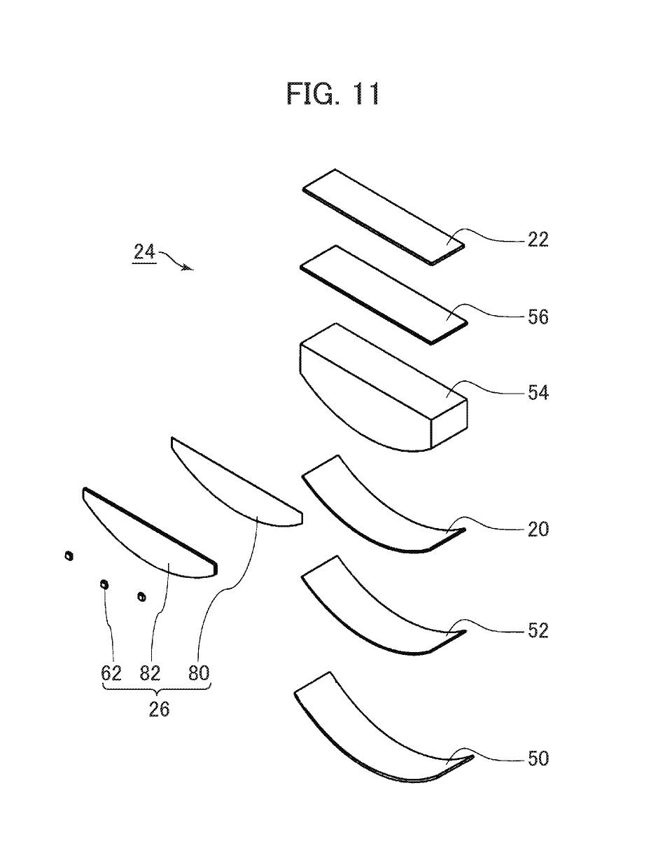

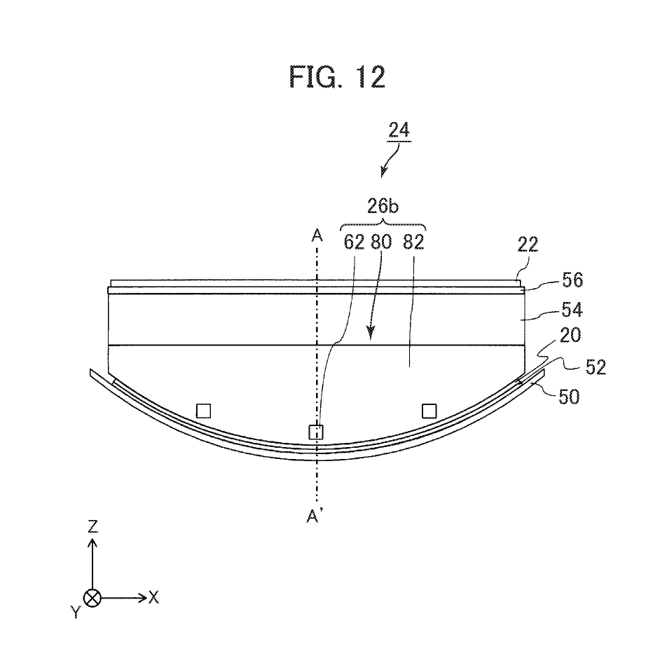

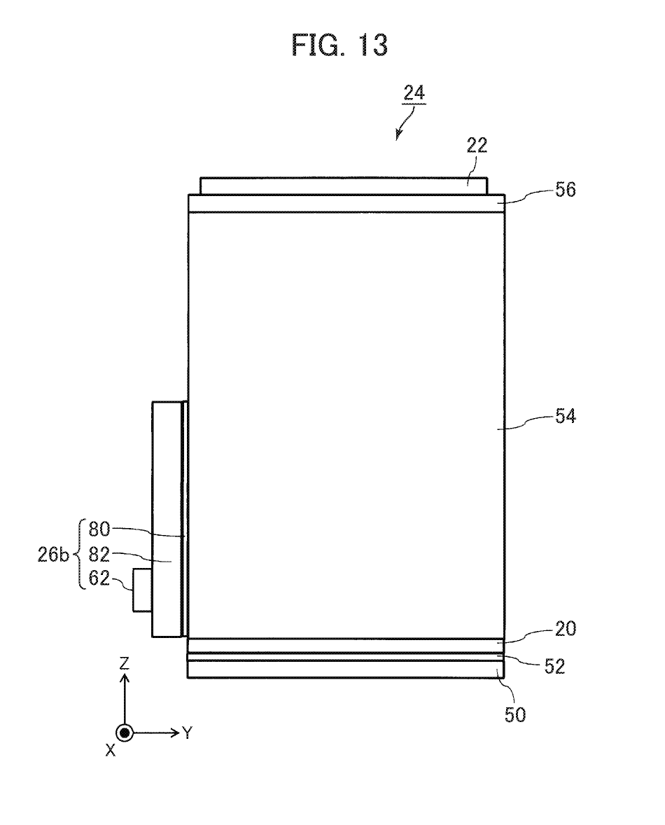

FIG. 10 shows a perspective view of the laminated block 24 and a temperature sensor unit 26b in the second embodiment. FIG. 11 shows an exploded perspective view of the laminated block 24 and the temperature sensor unit 26b. FIG. 12 shows a side view of the laminated block 24 and the temperature sensor unit 26b. FIG. 13 shows an A-A′ cross sectional view in FIG. 12.

Also in the second embodiment, the temperature sensor unit 26b is provided on the both long side surfaces of the laminated block 24.

As the configurations of the two temperature sensor units 26b are the same, one temperature sensor unit 26b will be described here. The temperature sensor unit 26b includes the thermistor 62, a metal plate 80 as a heat conduction member, and a side board 82. Note that in FIG. 10 to FIG. 13, plural thermistors 62 are shown, however, in the second embodiment, the number of the thermistors 62 may be one.

The metal plate 80 is a plate-shaped member formed of metal. As in the case of the metal film 60 in the first embodiment, it receives the heat from the wave transmission/reception surface via the laminated block 24, and conducts the heat to the thermistor 62. It is also preferable that the metal plate 80 is formed of a material with high thermal conductivity, and it is formed with a material with thermal conductivity higher than that of at least the respective members forming the laminated block 24. In the present embodiment, the metal plate 80 is formed with a copper plate or a graphite sheet. Further, as in the case of the metal film 60 in the first embodiment, the metal plate 80 is provided in the vicinity of the front-side (the wave transmission/reception surface side) edge of the side surface of the laminated block 24. Further, in the metal plate 80, at least the front-side end has a curved shape in accordance with the front-side edge of the backing member 54.

The side board 82 is provided with the thermistor 62 by soldering or the like. The side board 82 is laminated on the metal plate 80 so as to cover the metal plate 80. The side board 82 is connected to the metal plate 80 by bonding or the like. The side board 82 is provided with a through hole as a thermal conduction path for the metal plate 80 to conduct the heat received from the laminated block 24 to the thermistor 62. The through hole is a hole passing through the side board 82. The inner side of the hole is coated with metal. In the through hole, the metal-plate side end is in contact with the metal plate 80. With this configuration, the heat from the metal plate 80 is conducted to the through hole. The other end of the through hole is provided in the vicinity of the thermistor 62, and with this configuration, the heat of the metal plate 80 is conducted to the thermistor 62.

The metal plate 80 has the same functions as those of the metal film 60 in the first embodiment. That is, the metal plate 80 exerts the detection region expansion function of expanding the temperature detection region of the thermistor 62, and the thermal diffusion function of diffusing the heat of the wave transmission/reception surface of the probe 12. Further, the rear-side end of the metal plate 80 is not extended to the IC 22 as the main heat source, but is provided so as to cover about the half of the front side of the side surface of the backing member 54. With this configuration, the metal plate 80 exerts the detection region expansion function and the thermal diffusion function, while suppressing conduction of the heat from the IC 22 as the main heat source to the wave transmission/reception surface side.

As described above, the embodiments according to the present invention have been explained. However, the present invention is not limited to the above-described embodiments, but various changes can be made within a range not departing from the subject matter of the present invention.

### LIST OF REFERENCE SIGNS

10 . . . ultrasonic diagnostic device, 12 . . . probe, 14 . . . device main body, 20 . . . vibrator array, 22 . . . IC, 24 . . . laminated block, 26 . . . temperature sensor unit, 30 . . . control unit, 32 . . . wave transmission/reception condition setting unit, 34 . . . transmission/reception wave control unit, 36 . . . power consumption calculation unit, 38 . . . wave transmission/reception surface temperature estimation unit, 40 . . . warning control unit, 42 . . . transmission/reception unit, 44 . . . display processing unit, 46 . . . display unit, 48 . . . operation panel, 50 . . . protective layer, 52 . . . acoustic matching layer, 54 . . . backing member, 56 . . . relay board, 60 . . . metal film, 62 . . . thermistor, 80 . . . metal plate, 82 . . . side board.

1. An ultrasonic diagnostic device comprising:
an ultrasonic probe having: a laminated body including a vibrator array to transmit/receive an ultrasonic wave, an acoustic matching layer provided between an ultrasonic wave transmission/reception surface and the vibrator array, and a backing layer; and a temperature detection unit provided on at least one side surface of the laminated body; and
a temperature estimation unit that estimates a surface temperature of the wave transmission/reception surface based on a detection temperature detected with the temperature detection unit,
wherein the temperature detection unit includes:
a heat conduction member to receive heat from the laminated body, which is provided in the vicinity of an edge on the wave transmission/reception surface side of the side surface and which has a shape extending along the edge; and
a temperature sensor to detect a temperature of the heat conduction member.

2. The ultrasonic diagnostic device according to claim 1, wherein the heat conduction member exerts a detection region expansion function of expanding a temperature detection region of the temperature sensor, and a thermal diffusion function of diffusing heat of the wave transmission/reception surface.

3. The ultrasonic diagnostic device according to claim 1, wherein the heat conduction member extends in a strip in a direction along the edge.

4. The ultrasonic diagnostic device according to claim 1,
wherein the edge on the wave transmission/reception surface side of the side surface is curved, and
wherein an end at least on the wave transmission/reception surface side of the heat conduction member has a curved shape along the edge on the wave transmission/reception surface side of the side surface.

5. The ultrasonic diagnostic device according to claim 1, wherein the heat conduction member is formed with a metal film.

6. The ultrasonic diagnostic device according to claim 1,
wherein the temperature detection unit is a board connected to the heat conduction member, and further comprises a board including a thermal conduction path to conduct heat from the heat conduction member to the temperature sensor, and
wherein the temperature sensor is provided on the board.

7. An ultrasonic probe comprising:
a laminated body including a vibrator array to transmit/receive an ultrasonic wave, an acoustic matching layer provided between an ultrasonic wave transmission/reception surface and the vibrator array, and a backing layer; and
a temperature detection unit provided at least on one side surface of the laminated body,
wherein the temperature detection unit includes:
a heat conduction member to receive heat from the laminated body, which is provided in the vicinity of an edge on the wave transmission/reception surface side of the side surface, and which has a shape extending along the edge; and
a temperature sensor to detect a temperature of the heat conduction member.
